(19) United States
(12) Patent Application Publication
(10) Pub. No.: US 2007/0282170 A1
(43) Pub. Date: Dec. 6, 2007

(54) Rake Retractor and Needle Assembly for Minimally Invasive Surgical Applications

(76) Inventor: Sundaram Ravikumar, Briarcliff Manor, NY (US)

Correspondence Address: GORDON & JACOBSON, P.C., 60 LONG RIDGE ROAD, SUITE 407, STAMFORD, CT, 06902, US

(21) Appl. No.: 11/610,746

(22) Filed: Dec. 14, 2006

Related U.S. Application Data
(63) Continuation-in-part of application No. 11/420,927, filed on May 30, 2006.
(60) Provisional application No. 60/828,916, filed on Oct. 10, 2006.

(51) Int. Cl. A61B 1/32

(52) U.S. Cl. 600/211

(57) ABSTRACT

A rake retractor is provided which is preferably used in conjunction with a hollow needle having a diameter of 2.5 mm or smaller. The rake retractor broadly includes a shaft coupled to a plurality of free ends. The needle and/or rake retractor are preferably provided with a mechanism for locking the shaft of the rake retractor relative to the needle so that the rake retractor and needle can be fixed in place relative to each other. The free ends or prongs of the retractor at the end of the shaft are biased to an open position. When the rake rectractor is extended through the needle such that the free ends extend outside the needle, the free ends open up in a rake configuration. When the rake rectractor is pulled back relative to the needle such that the needle extends over the free ends, the free ends assume a closed position.

This application claims the benefit of provisional application Ser. No. 60/828,916 filed Oct. 10, 2006 and is a continuation-in-part of U.S. Ser. No. 11/420,927 filed May 30, 2006, both of which are incorporated herein in their entireties.

### BACKGROUND OF THE INVENTION

1. Field of the Invention

This invention relates broadly to surgical instruments. More particularly, this invention relates to a combination rake retractor and needle assembly. The invention has particular application to laparoscopic-type surgery, although it is not limited thereto.

2. State of the Art

Over the last two decades, minimally invasive surgery has become the standard for many types of surgeries which were previously accomplished through open surgery. Minimally invasive surgery generally involves introducing an optical element (e.g., a laparoscope or endoscope) through a surgical or natural port in the body, advancing one or more surgical instruments through additional ports or through the endoscope, conducting the surgery with the surgical instruments, and withdrawing the instruments and scope from the body.

In laparoscopic surgery (broadly defined herein to be any surgery where a port is made via a surgical incision, including but not limited to abdominal laparoscopy, arthroscopy, spinal laparoscopy, etc.), a port for a scope is typically made using a surgical trocar assembly. The trocar assembly often includes a port, a sharp pointed element (trocar) extending through and beyond the distal end of the port, and at least in the case of abdominal laparoscopy, a valve on the proximal portion of the port. Typically, a small incision is made in the skin at a desired location in the patient. The trocar assembly, with the trocar extending out of the port, is then forced through the incision, thereby widening the incision and permitting the port to extend through the incision, past any facia, and into the body (cavity). The trocar is then withdrawn, leaving the port in place. In certain circumstances, an insufflation element may be attached to the trocar port in order to insufflate the surgical site. An optical element may then be introduced through the trocar port. Additional ports are then typically made so that additional laparoscopic instruments, typically having rigid shafts may be introduced into the body.

Trocar assemblies are manufactured in different sizes. Typical trocar port sizes include 5 mm, 10 mm, and 12 mm (available from companies such as Taut and U.S. Surgical), which are selected to permit variously sized laparoscopic instruments to be introduced therethrough including, e.g., graspers, dissectors, staplers, scissors, suction/irrigators, clamps, forceps, biopsy forceps, etc. While 5 mm trocar ports are relatively small, in some circumstances where internal working space is limited (e.g., children), it is difficult to place multiple 5 mm ports in the limited area. In addition, 5 mm trocar ports tend to limit movements of instruments inside the abdominal cavity to a great extent and leave behind undesired scarring after surgery.

Of particular importance in abdominal surgery is the need for a surgeon to move and keep aside organs and other structures in the body cavity during an operation. In open surgery, rake retractors are used for this purpose. In laparoscopic surgery, laparoscopic rake retractors which fit through an associated trocar port and be able to function appropriately have not been commercially available.

U.S. Pat. No. 6,743,237 to Dhindsa describes a collapsible endoscopic rake. The rake of Dhindsa is described as being useful for collecting and withdrawing ureteral and calaceal stones and stone fragments from renal and biliary systems. A flexible sheath is provided with the rake into which the end elements of the rake can be withdrawn and which can be passed with the rake through a tortuous path (i.e., endoscopically). It will be appreciated that because the system of Dhindsa must be very flexible it is useful for brushing stones and stone fragments but could not be utilized as a laparoscopic rake retractor which could move and hold aside larger body structures such as organs.

Thus, there remains a need in the art to provide a laparoscopic rake retractor device which can function through a small diameter laparoscopic instrument. It also remains a need in the art to provide a laparoscopic type rake retractor device which can work in conjunction with extremely small ports.

### SUMMARY OF THE INVENTION

It is therefore an object of the invention to provide a rake retractor to assist surgeons in moving obstructive objects during operation procedures such as laparoscopic surgery.

It is an additional object of the invention to provide a minimally invasive surgical assembly having a rake retractor that is positioned into a 2.5 mm or smaller port device such as a laparoscopic needle.

It is another object of the invention to provide rake retractor prongs that are biased to an expanded position such that the prongs open outwardly as the rake retractor is extended out of an opening in the surgical assembly.

It is a further object of the invention to provide rake retractors having a variety of end configurations.

It is still another object of the invention to provide a method for affixing a rake retractor to a laparoscopic surgical instrument so that the rake retractor can be affixed in one position relative to the surgical assembly instrument.

In accord with these objects, which will be discussed in detail below, a rake retractor is provided which is preferably used in conjunction with a hollow needle having a diameter of 2.5 mm or smaller. The rake retractor according to the invention broadly includes a shaft coupled to a plurality of free ends. The shaft is preferably relatively rigid, and the shaft and/or needle are provided with means for locking the shaft relative to the needle so that the rake retractor and needle can be fixed in place relative to each other. The free ends or prongs of the retractor at the end of the shaft are biased to an open position. When the rake rectractor is extended through the needle such that the free ends extend outside the needle, the free ends open up in a rake configuration. When the rake rectractor is pulled back relative to the needle such that the needle extends over the free ends, the free ends assume a closed position.

More particularly, the rake retractor of the invention is designed so that it can be easily positioned through a laparoscopic needle. The rake retractor prongs remain bunched inside the needle in a collapsed position until the surgeon moves the retractor and needle relative to each other such that the retractor extends outside the distal end of the needle. When this extension occurs, the rake prongs are biased to an open position so that they automatically spread relative to each other into an expanded position as the prongs leave the interior of the needle. Similarly, when the surgeon moves the needle and retractor relative to each other such that the retractor is withdrawn back into the needle, the prongs progressively return to a collapsed position against their opening bias. By having the ability to lock the retractor relative to the needle, the surgeon can control the relative breadth of and rigidity assumed by the rake retractor prongs.

The prongs of the rake retractor may be affixed to the rake shaft by a simple weld or mechanical fixing mechanism. Alternatively, the prongs may be extensions of the shaft itself. The prongs themselves may be straight or have a variety of free end configurations. For example, the prongs may have one or more bends to assist the surgeon on reaching obstructed areas or the prong may be fitted with ball tips or tool-like structural configurations. Similarly, the prongs may have an arcuate shape whereby they form a curvature. Generally, the prongs are provided with blunted configurations to guard against the possibility of the surgeon pressing the instrument too hard against an internal body organ and thereby puncturing the organ.

In the preferred embodiments, the free ends of the rake prongs are positionally offset relative to one another so that when engaged to return to the collapsed position, the free ends do not simultaneously occupy the same lateral space. This offset feature allows the free ends of the rake prongs to be provided with end configurations of a size which would otherwise be unable to fit within the narrow confines of a laparoscopic needle or other passage when the prongs are in the collapsed position. Preferably, this offset feature is achieved by terminating the prongs (with or without attached free end configurations) at different positions relative to the longitudinal axis of the retractor shaft. It is not necessary that every rake prong is offset from the others; rather, it is important that enough rake prongs are offset so that the collapsed position inside the needle can be realized.

As previously mentioned, an important feature for the surgeon is to have the ability to affix the rake retractor in a position relative to the needle. This objective is achieved in the present invention by providing locking means to affix the shaft of the rake retractor to a position along the interior of the needle. This locking means may include a large number of mechanical embodiments such as screw mechanisms and peg-notch junctions to name a few.

Additional objects and advantages of the invention will become apparent to those skilled in the art upon reference to the detailed description taken in conjunction with the provided figures.

### BRIEF DESCRIPTION OF THE DRAWINGS

FIG. 1 shows the rake retractor in a first embodiment having wire prong free ends.

FIG. 2 shows the rake retractor having spoon-shaped configurations at the ends of the rake prongs.

FIG. 3 shows the rake retractor in another embodiment having ball-tip configurations at the ends of the rake prongs.

FIG. 4 is a representation of the rake retractor in which the ends of the rake prongs are in a rake configuration where they are bent near the free ends.

FIG. 5 is a representation of a rake retractor where the rake prongs each have bend points at two positions along each prong.

FIG. 6 is a representation of a rake retractor having prongs in an arcuate, curved shape.

FIG. 7 shows the rake retractor extended from a needle having a bend in the shaft of the retractor.

FIG. 8 is a drawing of the shaft of a retractor instrument slidably inserted through a needle. This figure also shows one locking means by which the retractor shaft can fixed in a position relative to the needle.

FIG. 9 shows a schematic section of the surgical system having the rake retractor in a collapsed position inside a needle.

### DETAILED DESCRIPTION OF THE PREFERRED EMBODIMENTS

Turning to FIG. 1, a rake retractor 10 includes a rake shaft 16 and a plurality of rake prongs 20 that extend from rake handle 16. The rake shaft has an outer diameter not greater than 2.5 mm (0.1 inches) and more preferably has an outer diameter of 1.5 mm (0.06 inches). These dimensions of rake retractor 10 allow use in a minimally invasive surgical instrument according to the invention. The rake shaft 16 is preferably relatively rigid (which is defined herein as being not capable of being inserted through a tortuous path) and it preferably is non-resilient (i.e., it will set if bent). The cross-sectional area of the rake shaft (i.e., the area defined by a cut through the shaft) is preferably greater than the sum of the cross-sectional areas of the rake prongs. Rake prongs 20 are preferably joined to the rake shaft 16 by a weld 18 or any other means. Alternatively, the rake prongs may be extensions of the rake shaft 16, in which case the cross-sectional areas of the rake shaft and the sum of the rake prongs can be the same. The rake prongs 20 are biased to an expanded position such that the prongs spread relative to each other and relative to a longitudinal axis, the axis defined by the length of rake shaft 16. When the rake prongs 20 in the expanded position are returned to the interior of the surgical instrument, they collapse against the bias so that they are substantially parallel to the longitudinal axis. The rake prongs 20 also have free ends that are positionally offset relative to the longitudinal axis of the rake retractor so that when engaged to return to the surgical instrument in a collapsed position, the free ends do not simultaneously occupy the same lateral space inside the instrument.

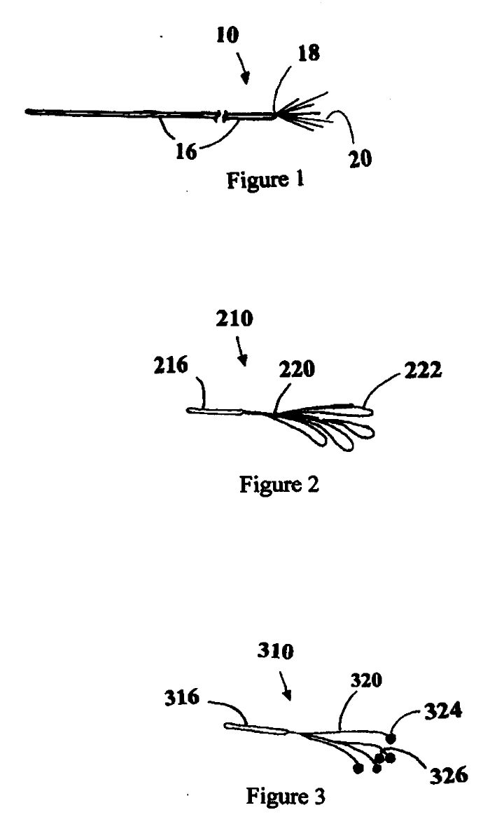

As shown in FIG. 2 in a second embodiment, a rake retractor 210 has a rake shaft 216 having the same dimensions as rake retractor 10 but includes a plurality of spoon-shaped elements 222 positioned at the distal end of prongs 220. These spoon-shaped elements can be used by the surgeon to scoop liquid or harness particulate materials away that would otherwise interfere in the surgical procedure. Alternatively, these elements may be used to assist the physician in the placement of desired surgical tools or pharmaceutical agents. Spoon-shaped elements 222 are blunt and prevent damage to tissue. Rake prongs 220 are biased to an expanded position such that the prongs spread relative to each other and relative to a longitudinal axis, the axis defined by the length of rake shaft 216. When rake prongs 220 in the expanded position are returned to the interior of the surgical instrument, they collapse against the bias so that they are substantially parallel to the longitudinal axis. Rake prongs 220 also have free ends that are positionally offset relative to the longitudinal axis so that when engaged to return to the surgical instrument in a collapsed position, spoon-shaped elements 222 do not simultaneously occupy the same lateral space inside the instrument.

As shown in FIG. 3 in a third embodiment, a rake retractor 310 has a similar rake shaft 316 and rake prongs 320 having ball-shaped end elements 324. Ball-shaped end elements 324 may be used by the surgeon to prod interfering materials aside or to move objects closer into a desired position. In addition, ball shaped end elements 324 are blunt and prevent damage to tissue. If desired, more than one ball-shaped element 324 may also extend from a single rake prong 326 (i.e., the prong can be branched). Rake prongs 320 are biased to an expanded position such that the prongs spread relative to each other and relative to a longitudinal axis, the axis defined by the length of rake shaft 316. When rake prongs 320 in the expanded position are returned to the interior of the surgical instrument, they collapse against the bias so that they are substantially parallel to the longitudinal axis. Rake prongs 320 also have free ends that are positionally offset relative to the longitudinal axis so that when engaged to return to the surgical instrument in a collapsed position, the ball-shaped elements 324 do not simultaneously occupy the same lateral space inside the instrument.

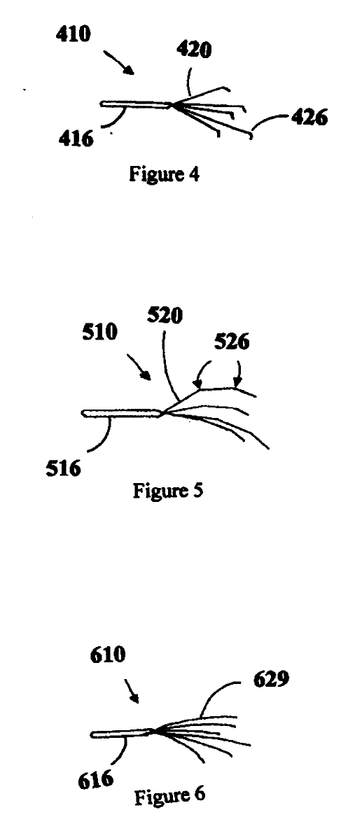

As shown in FIG. 4 in a fourth embodiment, rake retractor 410 has rake prongs 420 which include a bend 426 in each rake prong. These bends 426 may be positioned in one direction so that rake retractor 410 resembles a millimeter scale version of a traditional yard rake. Rake retractor 410 can then be used to scrape away obstructing objects in the surgeon's view. The same bias and positional offset characteristics as described in the first embodiment are also present in this fourth embodiment.

As shown in FIG. 5 in a fifth embodiment, rake retractor 510 may include two or more bends 526. These bends serve the same function as a described for above. However the inclusion of multiple bends may allow the surgeon to reach crevices in surgery that would otherwise be unreachable. The same bias and positional offset characteristics as described in the first embodiment are also present in this fifth embodiment.

As shown in FIG. 6 in a sixth embodiment, rake retractor 610 may include arch-shaped (arcuate) rake prongs 629. Like rake retractor 510, these arch-shaped rake prongs 629 provide the surgeon with a tool to reach crevices in surgery that may otherwise be unreachable with straight rake prongs. The same bias and positional offset characteristics as described in the first embodiment are also present in this sixth embodiment.

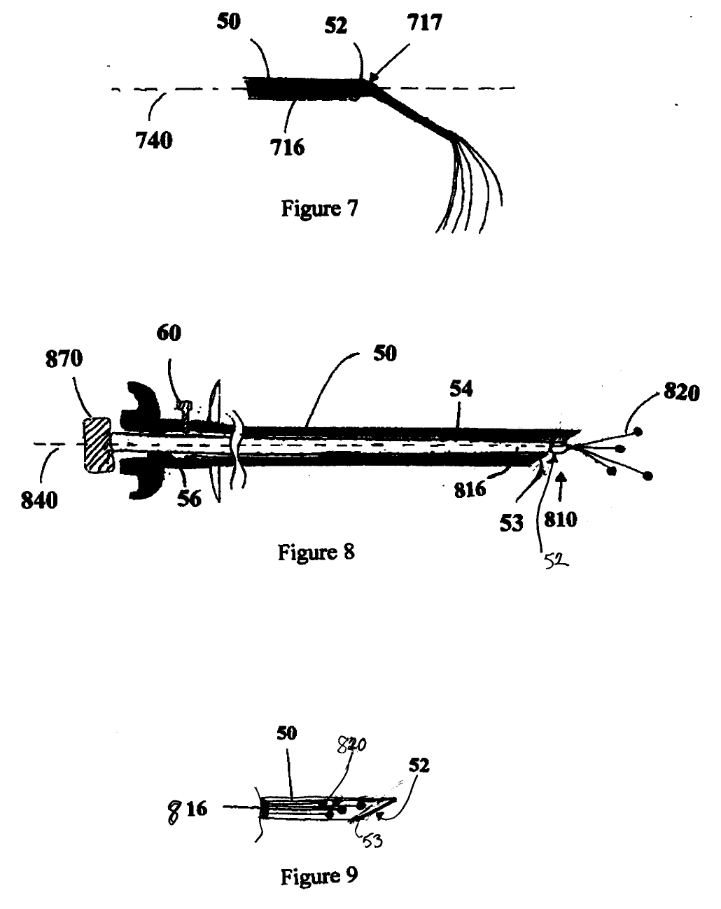

In still another embodiment of the invention, at least a portion of the rake shaft is resilient, and the rake retractor may have one or more bends 717 in shaft 716 as shown in FIG. 7. Bend 717 may assist the physician in reaching difficult areas below the skin during surgery in a manner similar to the arch shaped prongs of an earlier described embodiment. The same bias and positional offset characteristics as described in the first embodiment are also present in this sixth embodiment. In addition, shaft 716 has a distal end which is biased to an angled position relative to a longitudinal axis through the use of one or more bends 717.

In accord with any of the embodiments of FIGS. 1, 4, 5, 6, and 7, the ends or prongs of the rake retractor may be formed from wires. In preferred arrangements, where the tips of the prongs are formed from wires (as opposed to the arrangements of FIGS. 2 and 3), the wire of each prong is preferably twisted on itself in order to provide a blunt end and increased stiffness.

Turning now to FIGS. 8 and 9, the rake retractor 810 is preferably used in a combination with a surgical needle 50 having a maximum diameter of, e.g., 2.5 mm, through which it coaxially extends. Needle 50 is shown as being hollow with a sharp distal end 53 defining an opening 52. According to one aspect of the preferred embodiment of the invention, rake retractor 810 and needle 50 are sized so that at least a portion of rake shaft 816 of rake retractor 810 interferingly slides against the inner surface of the needle 50, thereby forming a seal at 54 which is effective against desufflation of the abdominal cavity. The small difference between the inner diameter of needle 50 and the rake shaft 816 of rake retractor 810 results in a sliding interference fit which can be felt as a drag. If desired, only a portion of shaft 816 is sized to interferingly slide against the inner surface of the needle. In addition, or in lieu of the sliding interference fit, the needle 50 may include an internal gasket or seal 56 which seals against the outer diameter of rake shaft 816 and may provide a similarly described interfering sliding effect. The needle 50 and/or the rake retractor 810 are also preferably provided with means for affixing the rake retractor 810 relative to needle 50 into a locked position. In FIG. 8, the needle is shown with a thumb screw 60 which can be used to lock the rake retractor 810 relative to the needle 50. The rake retractor 810 is also shown with a handle 870 by which the rake retractor can be manipulated. The longitudinal axis of the rake retractor is indicated as 840, and rake prongs 820 are shown having ball-shaped end elements as in the embodiment of FIG. 3, although the rake retractor/needle combination applies to all of the rake retractors previously described.

Use of the rake retractor 810 with the needle 50 provides various desirable results. First, as described hereinafter, the rake retractor can be introduced with the needle rather than through a larger trocar port. Second, because needle 50 has a sharp beveled end, if the prongs of the rake retractor are biased in two dimensions (i.e., they spread out in an x dimension and down or up in a y dimension), the rotation of the rake retractor relative to the needle can be used to control the y dimension movement of the rake prongs. More particularly, if the rake retractor is located so that the prongs start at the most proximal portion of the needle opening, when the prongs are biased toward the side of the needle with the more proximal bevel location, the prongs will not be at all supported by the needle, whereas when the prongs are biased toward the side of the needle where the bevel tip extends the furthest, the prongs will be supported by the end of the needle and will not bend as much. Third, because the needle 50 is relatively rigid, the needle 50 and rake retractor 810 can be locked or affixed together, and together can transmit sufficient force to hold an organ in a desired location.

The method of the claimed invention is initiated when the operating surgeon obtains a hollow needle 50 having an outer diameter preferably not greater than 2.5 millimeters. The surgeon then inserts a sharp distal portion 53 of needle 50 into and through the skin of a patient using the sharp distal end in order to make a port. This insertion is preferably made with the rake retractor already in the needle with the prongs located inside the needle. Alternatively, the rake retractor may be inserted into the needle after the needle has been used to make a port. In any event, the surgeon may then extend the prongs of the rake retractor out of the needle by moving the needle and retractor relative to each other so that the prongs of the retractor extend beyond the distal end of needle.

As discussed above, the rake prongs are constructed so that they are biased to an open position. As the prongs slide outside an exit opening 52 of surgical needle 50, they automatically extend into partially and then fully expanded positions as shown in FIGS. 1-8.

At any time, the surgeon may lock the rake retractor relative to the needle using the thumb screw 60. When the rake retractor is fixed to the needle, the surgeon can maneuver the needle so that rake prongs of rake retractor contact objects in a cavity (e.g., the abdomen) beneath the skin. If it is desired to reposition the rake retractor relative to the needle, screw 60 may be loosened. The rake retractor and/or the needle can then be moved or repositioned as needed by the surgeon.

If it is desired to remove the rake retractor from the cavity, the retractor should be pulled proximally relative to the needle 50 such that the prongs of the retractor are forced back into the opening 52 of the needle. Force may be required so that the rake prongs will bend against their bias. Upon full retraction into needle 50, prongs rest in a collapsed position, e.g., as shown in FIG. 9. The collapsed position of FIG. 9 is made possible by the longitudinal offset nature of the ball elements 824 provided at the ends of prongs 820. Without the longitudinal offset, the end configurations would attempt to occupy the same lateral area within needle 50 and would have a cumulative diameter greater than the lateral dimension provided by the needle.

As previously mentioned, the surgical system of the invention includes a locking means that is used to fix the relative location of rake retractor to needle 50. In the embodiment of FIG. 8, the locking means is a thumb screw 60 in conjunction with a thread (not shown) on the needle shaft 58. As aforementioned, the thumb screw, when screwed through the needle shaft, engages the shaft 816 of the needle retractor and thus locks the rake retractor 810 at a fixed position relative to the needle 50.

In a second example of the locking means, one or more threaded radial bores (not shown) are located on the needle shaft 58 of needle 50, notches (now shown) are provided on the shaft of the retractor, and a screw or peg 60 is provided which extends through one of the threaded radial bores in the needle or its handle. When it is desired to fix the rake retractor in a position relative to needle 50, the screw or peg 60 is engaged into a notch. When it is desired to release needle 50, screw or peg 60 is loosened so that it is no longer engages the notch. It will be appreciated that instead of using a screw or peg-like mechanism, that a spring-loaded pin which extends through a radial hole in the needle can be used to lock the needle shaft 58 or the rake retractor relative to needle 50.

A third example of a locking means includes radial grooves (not shown) on the shaft of the rake retractor and a clip (not shown) having spring arms and a clip shaft. The clip shaft extends through a wall of the needle, or more preferably, its handle, and the spring arms engage a radial groove (not shown) on the rake retractor shaft. When the shaft of rake retractor is pushed or pulled relative to the needle, the spring arms spread to permit movement of the rake retractor shaft past the clip. It will be appreciated that if the spring arms are sufficiently springy, grooves are not required on the shaft as the spring arms will firmly hold the shaft in position.

A fourth locking means may include a plastic screw which extends around the shaft of rake retractor, and an inner thread (not shown) located on a handle or knob of needle 50. When it is desired to fix rake retractor relative to needle 50, the plastic screw is screwed into the threaded handle or knob needle of needle 50. The plastic screw and the inner thread of the handle or knob of needle 50 are sized to cause the plastic screw to deform and tighten around the rake retractor shaft when the screw is screwed into the thread, thereby fixing the locations of the needle 50 and the rake retractor relative to each other. When it is desired to release the rake retractor, the plastic screw is unscrewed sufficiently to permit movement of the rake retractor relative to the needle. As will be appreciated by those skilled in the art, the plastic screw may have a gripping member such as a head to help the practitioner apply torque.

A fifth locking means involves a cam element (not shown) rotatably coupled to a handle of needle 50 and a pin. When in a first orientation, the cam element permits a rear portion of the shaft of the rake retractor to move in an uninhibited manner. When in a second orientation, the cam element engages a rear portion of the shaft and holds it fixed relative to needle 50.

Details of the different locking means may be seen with reference to previously incorporated parent application Ser. No. 11/420,927. Numerous other locking means and their equivalents will be evident to one of ordinary skill in the art upon a brief review of the means described herein.

There have been described and illustrated herein several embodiments of a rake retractor, surgical assembly, and methods for the use thereof. While particular embodiments of the invention have been described, it is not intended that the invention be limited thereto, as it is intended that the invention be as broad in scope as the art will allow and that the specification be read likewise. Thus, while particular materials for configurations for the rake retractor the needle and surgical instrument have been disclosed, it will be appreciated that other materials may be used as well. Also, different aspects of different embodiments can be combined. For example, any of prong ends of FIG. 1-3 can be used in conjunction with any of the prong bends of FIGS. 4-6. Likewise, the bent shaft of FIG. 7 can be used in conjunction with any of the arrangements of FIGS. 1-6, or any combination thereof. In addition, while particular locking elements and systems have been disclosed for fixing the rake retractor relative to the needle, it will be understood that other mechanisms can be used. For example, and not by way of limitation, a latch-catch system can be used. It will therefore be appreciated by those skilled in the art that yet other modifications could be made to the provided invention without deviating from its spirit and scope as claimed.

What is claimed is:

1. A rake retractor for use within a hollow surgical instrument having an inner diameter less than 2.5 mm, the instrument having a distal end and an interior, comprising:
a) a relatively rigid shaft having a proximal end, a distal end, a longitudinal axis, and a diameter less than 2.5 mm; and
b) a plurality of rake prongs each including a first end coupled to said distal end of said shaft and an opposite free end, said free end of at least two of said rake prongs terminating at differing lengths relative to the longitudinal axis, said rake prongs biased to spread relative to each other into an expanded position,
wherein when said rake retractor is inserted into the surgical instrument, said rake prongs are structured to collapse against said bias to reside within the interior,
and when said rake retractor is extended out of the distal end of the instrument, said rake prongs move into said expanded position.

2. The rake retractor of claim 1, wherein:
said plurality of rake prongs are joined to the shaft by a weld.

3. The rake retractor of claim 1, wherein:
said shaft has at least one bend.

4. The rake retractor of claim 1, wherein:
at least one of said plurality of rake prongs is a wire.

5. The rake retractor of claim 1, wherein:
said rake prongs have respective free ends, said free ends terminating at two or more positions along the longitudinal axis.

6. The rake retractor of claim 5, wherein:
at least one of said free ends has a blunted end configuration.

7. The rake retractor of claim 6, wherein:
said blunted end configuration includes one of a ball-tip shape and a spoon-shape.

8. The rake retractor of claim 5, wherein:
combined diameters of said free ends is greater than an inner diameter of the surgical instrument.

9. The rake retractor of claim 1, wherein:
said shaft comprises a proximal extension of at least one of said rake prongs.

10. The rake retractor of claim 1, further comprising:
locking means for affixing said rake retractor to the surgical instrument.

11. A surgical system, comprising:
a) a sharp hollow needle having an outer diameter less than or equal to 2.5 mm; and
b) a rake retractor positioned within the hollow surgical instrument, said rake retractor including:
i) a shaft having a proximal end and a distal end; and
ii) a plurality of rake prongs including a first end coupled to said distal end of said shaft and an opposite free end, said free end of at least two of said rake prongs terminating at differing lengths relative to a longitudinal axis, said rake prongs biased to spread relative to each other into an expanded position, such that in a first position said rake prongs spread into an expanded position outside of said surgical instrument and in a second position said rake prongs reside in a collapsed position within an interior of said surgical instrument.

12. The surgical system of claim 11, wherein:
said shaft of said rake retractor is relatively rigid.

13. The surgical system of claim 11, wherein:
at least one of said plurality of rake prongs are joined to said shaft by a weld.

14. The surgical system of claim 11, wherein:
said shaft has at least one bend.

15. The surgical system of claim 11, wherein:
at least one of said plurality of rake prongs is a wire.

16. The surgical system of claim 11, wherein:
said rake prongs have respective free ends, said free ends terminating at two or more positions along the longitudinal axis.

17. The surgical system of claim 16, wherein:
combined diameters of said free ends is greater than an inner diameter of said surgical instrument.

18. The surgical system of claim 16, wherein:
at least one free end includes one of a ball-tip configuration, a spoon-shaped configuration, and a blunted end configuration.

19. The surgical system of claim 11, including:
locking means for affixing the rake retractor to said hollow surgical instrument.

20. A surgical method, comprising:
a) obtaining a hollow needle having an outer diameter not greater than 2.5 millimeters and a sharp distal end, and a rake retractor having a rake shaft which extends through the hollow needle and also having rake prongs at a first end of the rake shaft, the rake prongs biased to an open position, the rake prongs terminating at differing lengths relative to a longitudinal axis, and the rake retractor being moveable relative to the hollow needle;
b) inserting a distal portion of the hollow needle into the skin of a patient using the sharp distal end, the insertion made with the rake retractor in a closed position; and
c) moving one of the surgical instrument and the needle relative to the other such that the rake prongs extend out of the needle and open relative to each other.

21. The surgical method of claim 20, further comprising:
maneuvering the rake to contact an organ beneath the skin.

22. The surgical method of claim 20, further comprising:
fixing the rake and the needle relative to each other.
